(19) United States
(12) Patent Application Publication
(10) Pub. No.: US 2026/0295019 A1
(43) Pub. Date: Oct. 1, 2026

(54) USES OF SUPER-OXIDE DISMUTASE SUPPLEMENTS

(71) Applicant: ADVANCE PHARMACEUTICAL, INC., Holtsville, NY (US)

(72) Inventor: Liaquat HOSSAIN, Holtsville, NY (US)

(21) Appl. No.: 19/476,854

(22) PCT Filed: Apr. 19, 2024
(86) PCT No.: PCT/US2024/025371

§ 371 Date: Oct. 20, 2025

Related U.S. Application Data
(60) Provisional application No. 63/497,500, filed on Apr. 21, 2023.

(51) Int. Cl. A61K 38/44; A61K 31/718; A61K 36/899; A61P 3/10; A61P 17/10; A61P 25/28; A61P 39/06

(52) U.S. Cl. CPC A61K 38/446; A61K 31/718; A61K 36/899; A61P 3/10; A61P 17/10; A61P 25/28; A61P 39/06; C12Y 115/01001

(57) ABSTRACT

Provided are methods of use of compositions comprising super-oxide dismutase and a soluble fiber for treatment or prevention of disorders. In certain aspects, the compositions can be used for: 1) protection of tight junctions in Human Brain Microvascular Endothelial Cells (HBMEC) under hypoxic conditions; 2) reduction of A1C protein levels and fasting glucose in diabetes patients; and 3) reduction of inflammatory markers in acne patients.

### FIELD OF THE DISCLOSURE

The present disclosure relates generally to uses of compositions comprising super-oxide dismutase and a soluble fiber. The compositions can be used for the treatment or prevention of disorders associated with exposure to radical oxygen species. Among other uses, the compositions can be used for: 1) protection of tight junctions in Human Brain Microvascular Endothelial Cells (HBMEC) under hypoxic conditions; 2) reduction of A1C protein levels and fasting glucose in diabetes patients; and 3) reduction of inflammatory markers in acne patients.

### BACKGROUND OF THE DISCLOSURE

Superoxide dismutases (SODs) are a group of metalloenzymes that protect against cellular damage by reactive oxygen species (ROS). SOD catalyzes the dismutation of superoxide anion free radical (O₂⁻) into molecular oxygen and hydrogen peroxide (H₂O₂). ROS in cells can damage nucleic acids, proteins and lipids leading to decreased cellular function and possible apoptosis. Thus, the ability to convert ROS into harmless molecules is crucial for protecting cellular function and overall health.

While almost all organisms naturally produce some type of SOD, the levels of SOD produced in cells falls as the subject ages or when the subject is afflicted with certain health disorders. Also, the presence of various pollutants and toxins in the environment can cause increased levels of ROS in cells. Dietary supplementation of naturally produced SOD can therefore be important to maintaining health.

Another important dietary component is soluble fiber. Soluble fiber absorbs water and forms a gel in the digestive tract. Soluble fiber has many benefits in the gut, including slowing the digestion of certain types of lipids and carbohydrates, helping to prevent uptake of dietary cholesterol and preventing spikes in blood sugar levels. Soluble fiber is also important for maintaining a health gut microbiome, as gut bacteria can ferment some types of soluble fiber. Modern diets are typically low in soluble fiber and supplementation is often needed for improved health.

Antioxidants have been shown to have beneficial effects in the human body in combating free radicals, which are involved in many disease processes. However, the absorption of the effective metabolites of many antioxidants, such as polyphenols, depends on microbial metabolism of the antioxidants in the gut. Thus, for many nutritional supplements, these effective polyphenol metabolites are not produced and the antioxidant activity the supplement is limited.

### BRIEF SUMMARY OF THE DISCLOSURE

One aspect of the present disclosure provides a method of maintaining tight junctions between cells in a tissue exposed to hypoxic conditions in a subject comprising administering to the subject a composition comprising: a) from about 10 units to about 200 units superoxide dismutase; and b) from about 500 mg to about 8000 mg soluble fiber.

In embodiments of the method, the tissue exposed to hypoxic conditions comprises the blood brain barrier. In embodiments, the tissue comprises human brain microvascular endothelial cells. In embodiments, the hypoxic conditions are caused by a stroke, heart attack or accident.

Another aspect of the present disclosure provides a method of preventing or reducing symptoms associated with a neurological disorder in a subject comprising administering to the subject a composition comprising: a) from about 10 units to about 200 units superoxide dismutase; and b) from about 500 mg to about 8000 mg soluble fiber.

In embodiments of the method, the neurological disorder is Parkinson Disease (PD), Multiple Sclerosis (MS), or Alzheimer's Disease (AD).

Another aspect of the present disclosure provides a method of reducing fasting glucose in a subject having diabetes comprising administering to the subject a composition comprising: a) from about 10 units to about 200 units superoxide dismutase; and b) from about 500 mg to about 8000 mg soluble fiber.

In embodiments of the method, the fasting glucose is reduced in the subject by at least about 30%.

Another aspect of the present disclosure provides a method of reducing hemoglobin A1C level in a subject having diabetes comprising administering to the subject a composition comprising: a) from about 10 units to about 200 units superoxide dismutase; and b) from about 500 mg to about 8000 mg soluble fiber.

In embodiments of the method, the hemoglobin A1C level is reduced by at least about 15%.

In embodiments of the methods where the subject has diabetes, the subject has type 2 diabetes.

Another aspect of the present disclosure provides a method of reducing acne symptoms in a subject comprising administering to the subject a composition comprising: a) from about 10 units to about 200 units superoxide dismutase; and b) from about 500 mg to about 8000 mg soluble fiber.

In embodiments of the method, the acne symptoms are comedos, pimples or skin discoloration.

In embodiments of any of the methods described herein, the composition comprises from about 50 units to about 150 units superoxide dismutase. In embodiments, the composition comprises from about 70 units to about 100 units superoxide dismutase. In embodiments, the composition comprises from about 1000 mg to about 5000 mg soluble fiber. In embodiments, the composition comprises from about 2000 mg to about 4000 mg soluble fiber.

In embodiments of any of the methods described herein, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of any of the methods described herein, the ratio of superoxide dismutase to soluble fiber in the composition is from about 1:100 to about 1:1000 by weight. In embodiments, the ratio of superoxide dismutase to soluble fiber in the composition is from about 1:500 to about 1:700 by weight.

In embodiments of any of the methods described herein, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments of any of the methods described herein, the composition is in the form of a gel. Suitable gel compositions are described herein.

In embodiments of any of the methods described herein, the composition is in the form of a liquid. Suitable gel compositions are described herein.

In embodiments of any of the methods described herein, the composition is in the form of a powder. Suitable powder compositions are described herein.

### BRIEF DESCRIPTION OF THE DRAWINGS

FIG. 1 is a plot of hypoxia induced permeability as determined in an in vitro assay of a monolayer of human microvascular endothelial cells as described in Example 1. Basal represents the permeability of the monolayer under normal conditions and results are scaled so that basal permeability is 100%. The cell monolayer Cells were treated with the following agents: 1. No treatment, 2. Hypoxia induced control, 3. Hypoxia plus Fibersol-2, 4. Hypoxia plus SOD, 5. Hypoxia plus polyphenols [fruit juices], 6. Hypoxia plus finished product [SOD, Fibersol-2, polyphenols].

FIGS. 2A-F show imaging of cells exposed to antibody to ZO-1 as described in Example 1. FIG. 2A—untreated control; FIG. 2B—hypoxia negative control (no treatment); FIG. 2C—treatment with prebiotic fiber only; FIG. 2D—treatment with Superoxide Dismutase only; FIG. 2E—treatment with polyphenols (fruit juice) only; FIG. 2F—treatment with SOD gel finished product.

FIGS. 3A-F show imaging of cells exposed to antibody to Occludin as described in Example 1. FIG. 3A—untreated control; FIG. 3B—hypoxia negative control (no treatment); FIG. 3C—treatment with prebiotic fiber only; FIG. 3D—treatment with Superoxide Dismutase only; FIG. 3E—treatment with polyphenols (fruit juice) only; FIG. 3F—treatment with SOD gel finished product.

FIGS. 4A-F show imaging of cells exposed to antibody to E-Cadherin as described in Example 1. FIG. 4A—untreated control; FIG. 4B—hypoxia negative control (no treatment); FIG. 4C—treatment with prebiotic fiber only; FIG. 4D—treatment with Superoxide Dismutase only; FIG. 4E—treatment with polyphenols (fruit juice) only; FIG. 4F—treatment with SOD gel finished product.

FIGS. 5A and 5B are before (5A) and after (5B) images of a subject treated with SOD gel (Revivify) and oral doxycycline as described in Example 3.

FIGS. 6A and 6B are before (6A) and after (6B) images of a subject treated only with oral doxycycline as described in Example 3.

### DETAILED DESCRIPTION OF THE DISCLOSURE

The present disclosure provides methods of use of compositions comprising superoxide dismutase and a soluble fiber. The compositions disclosed herein can be used as supplements to increase the levels of superoxide dismutase and soluble fiber in a subject. As described herein, the compositions can also comprise additional components such as antioxidants, vitamins or other nutrients along with excipients and other formulation agents.

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict from any reference cited herein and the specific teachings of this specification shall be resolved in favor of the latter. Likewise, any conflict from an art-understood definition of a word or phrase and a definition of the word or phrase as specifically taught in this specification shall be resolved in favor of the latter.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

Technical and scientific terms used herein have the meaning commonly understood by one of skill in the art to which the present application pertains, unless otherwise defined. Reference is made herein to various methodologies and materials known to those of skill in the art.

The term “superoxide dismutase” (sometimes abbreviated SOD) as used herein refers an enzyme that catalyzes the dismutation of superoxide anion free radical (O₂⁻) into molecular oxygen and hydrogen peroxide (H₂O₂). Superoxide dismutase has the Enzyme Classification (EC) number 1.15.1.1. Superoxide dismutase is an important component in protecting cells against damage from reactive oxygen species (ROS) such as O₂⁻ that are formed during cellular metabolism of oxygen. Superoxide dismutase thus acts as an important antioxidant and is present in almost all types of cellular organisms. However, particularly in complex organisms such as mammals, cellular levels of superoxide dismutase decrease as the organism ages, making the organism more susceptible to cellular damage from reactive oxygen species.

The term “soluble fiber” as used herein refers to any type of water soluble dietary fiber. When consumed, soluble fiber absorbs water to form a gel in the gut of the organism that helps to slow the metabolism of lipids and carbohydrates. Soluble fiber is also a prebiotic that can be fermented by gut bacteria which helps maintain a healthy gut microbiome.

The term “antioxidant” as used herein refers to a substance that significantly decreases the adverse effects of reactive species, such as reactive oxygen and nitrogen species by wholly or partially neutralizing reactive species. Antioxidants can be classified as “primary antioxidants” and “secondary antioxidants.” Primary antioxidants delay or inhibit the initiation step of oxidation, while secondary antioxidants slow down the oxidation by removing the substrate or by quenching free oxygen radicals.

### Methods of Use

In embodiments, present disclosure provides a method of maintaining tight junctions between cells in a tissue exposed to hypoxic conditions in a subject comprising administering to the subject a composition comprising: a) from about 10 units to about 200 units superoxide dismutase; and b) from about 500 mg to about 8000 mg soluble fiber.

Tight junctions (also called zonula occludens or occluding junctions) are a type of cell junction formed as an adhesion complex between two neighboring cells that provides a tight seal between the cells. Tight junctions are essentially a protein complex between two cells. In some embodiments, tight junctions may comprise one or more of the proteins Zonula occludens-1 (ZO-1), Occludin, Claudin-1 and E-Cadherin.

In embodiments of the method, the tissue exposed to hypoxic conditions comprises the blood brain barrier (BBB). The BBB is a network of blood vessels and tissue that is made up of closely spaced cells which helps keep harmful substances from reaching the brain. The blood-brain barrier lets some substances, such as water, oxygen, carbon dioxide, and general anesthetics, pass into the brain. It also keeps out bacteria and other substances.

In embodiments, the tissue comprises human brain microvascular endothelial cells (HBMECs). HBMECs are the main component of the human BBB. Tight junctions between HBMECs help form the BBB and prevent harmful substances carried in the blood from entering brain tissue. Inflammation and oxidative stress can cause loss of integrity of the BBB.

In embodiments, the hypoxic conditions are caused by a stroke, heart attack or accident. Hypoxic conditions of the brain occur when the flow of oxygen to the brain is impeded. In embodiments, the hypoxic conditions are brought on by a stroke. In embodiments, the hypoxic conditions are brought on by heart attack. In embodiments, the hypoxic conditions are brought on by accidents such as choking, drowning or head injury.

In embodiments, the present disclosure provides a method of preventing or reducing symptoms associated with a neurological disorder in a subject comprising administering to the subject a composition comprising: a) from about 10 units to about 200 units superoxide dismutase; and b) from about 500 mg to about 8000 mg soluble fiber.

In embodiments of the method, the neurological disorder is Parkinson Disease (PD), Multiple Sclerosis (MS), or Alzheimer's Disease (AD). Research has indicated that subjects with neurological diseases such as PD, MS and AD experience changes in their BBB, which can allow harmful molecules to enter brain tissue. In embodiments, the compositions described herein help maintain tight cell junctions and maintain the integrity of the BBB in subjects having neurological diseases. Helping to maintain the integrity of the BBB can help prevent or reduce symptoms associated with such neurological diseases.

In embodiments, the symptoms associated with PD are one or more of: 1) tremors in hands, arms, legs jaw or head; 2) muscle stiffness, including muscle contractions for an extended period of time; 3) slowness of movement; and 4) impaired balance and coordination.

In embodiments, the symptoms associated with MS are one or more of: 1) Loss of vision; 2) Double vision; 3) Vertigo; 4) Weakness in one or more limbs; 5) Facial weakness; 6) Facial pain (trigeminal neuralgia); 7) Numbness and tingling; 8) Muscle stiffness (spasticity); 9) Gait abnormalities and imbalance; 10) Cognitive problems; 11) Depression; 12) Fatigue; 13) Bowel and bladder problems; 14) Sexual dysfunction; and 15) Pain.

In embodiments, the symptoms associated with AD are one or more of: 1) memory loss; 2) poor judgment; 3) wandering; 4) losing things; 5) difficult completing daily tasks; 6) mood and personality changes; and 7) increased anxiety and/or aggression.

In embodiments, the present disclosure provides a method of reducing fasting glucose in a subject having diabetes comprising administering to the subject a composition comprising: a) from about 10 units to about 200 units superoxide dismutase; and b) from about 500 mg to about 8000 mg soluble fiber.

Fasting blood glucose (sugar) level is a well-known test for diabetes that reflects the presence of too much sugar in the blood of diabetes and pre-diabetic patients. In embodiments, a fasting blood glucose level less than 100 mg/dL (5.6 mmol/L) is normal. In embodiments, a fasting blood glucose level from 100 to 125 mg/dL (5.6 to 6.9 mmol/L) is considered prediabetes. In embodiments, a fasting blood glucose level of 126 mg/dL (7 mmol/L) or higher indicates diabetes.

In embodiments of the method, the fasting glucose is reduced in the subject by at least about 30%. In embodiments, the fasting glucose is reduced in the subject by at least about 10%, 15%, 20%, 25%, 30%, 35%, 40%, 45% or 50%.

Another aspect of the present disclosure provides a method of reducing hemoglobin A1C level in a subject having diabetes comprising administering to the subject a composition comprising: a) from about 10 units to about 200 units superoxide dismutase; and b) from about 500 mg to about 8000 mg soluble fiber.

A blood test for hemoglobin A1C is indicative of average blood sugar over a longer time period, e.g., 2 or 3 months. The test measures the percentage of blood sugar attached to hemoglobin, the oxygen-carrying protein in red blood cells. An A1C test can also be called a glycated hemoglobin test.

In general, higher the blood sugar levels lead to more hemoglobin with sugar attached. In embodiments, an A1C level of 6.5% or higher indicates that the subject has diabetes. In embodiments, an A1C level between 5.7% and 6.4% indicates that the subject has prediabetes. In embodiments, an A1C of below 5.7% is considered normal, i.e., non-diabetic.

In embodiments of the method, the hemoglobin A1C level is reduced by at least about 15%. In embodiments, the A1C level is reduced in the subject by at least about 10%, 15%, 20%, 25%, 30%, 35%, 40%, 45% or 50%.

In embodiments, the subject has type 2 diabetes. In embodiments, the subject has type 1 diabetes.

Another aspect of the present disclosure provides a method of reducing acne symptoms in a subject comprising administering to the subject a composition comprising: a) from about 10 units to about 200 units superoxide dismutase; and b) from about 500 mg to about 8000 mg soluble fiber.

In embodiments of the method, the acne symptoms are comedos (clogged hair follicles), pimples or skin discoloration.

In embodiments of any of the methods described herein, the composition used in the method is in the form of a gel. Suitable gel compositions are described herein.

In embodiments of any of the methods described herein, the composition used in the method is in the form of a liquid. Suitable gel compositions are described herein.

In embodiments of any of the methods described herein, the composition used in the method is in the form of a powder. Suitable powder compositions are described herein.

In embodiments of the above methods, the composition used comprises from about 10 units to about 200 units superoxide dismutase. In embodiments, the composition comprises comprising from about 50 units to about 150 units superoxide dismutase. In embodiments, the composition comprises from about 70 units to about 100 units superoxide dismutase. In embodiments, the composition comprises from about 20 units to about 190 units superoxide dismutase. In embodiments, the composition comprises from about 30 units to about 180 units superoxide dismutase. In embodiments, the composition comprises from about 40 units to about 170 units superoxide dismutase. In embodiments, the composition comprises from about 50 units to about 160 units superoxide dismutase. In embodiments, the composition comprises from about 60 units to about 150 units superoxide dismutase. In embodiments, the composition comprises from about 70 units to about 140 units superoxide dismutase. In embodiments, the composition comprises from about 80 units to about 130 units superoxide dismutase. In embodiments, the composition comprises from about 90 units to about 130 units superoxide dismutase. In embodiments, the composition comprises from about 75 units to about 95 units superoxide dismutase. In embodiments, the composition comprises from about 80 units to about 90 units superoxide dismutase. In embodiments, the composition comprises about 20, 30, 40, 50, 60, 70, 80, 90, 100, 110, 120, 130, 140, 150, or 160 units superoxide dismutase. In embodiments, the composition comprises about 84 units superoxide dismutase.

In embodiments of the above methods, the composition used comprises from about 1000 mg to about 5000 mg soluble fiber. In embodiments, the composition comprises from about 2000 mg to about 4000 mg soluble fiber. In embodiments, the composition comprises from about 1000 mg to about 10000 mg soluble fiber. In embodiments, the composition comprises from about 2000 mg to about 9000 mg soluble fiber. In embodiments, the composition comprises from about 3000 mg to about 8000 mg soluble fiber. In embodiments, the composition comprises from about 2000 mg to about 5000 mg soluble fiber. In embodiments, the composition comprises about 500, 1000, 1500, 2000, 2500, 3000, 3500, 4000, 4500 or 5000 mg soluble fiber.

In embodiments of the above methods, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

In embodiments of the above methods, the composition can comprise one or more superoxide dismutases as disclosed herein. In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the above methods, the composition can comprise one or more soluble fiber as disclosed herein. In embodiments, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments of any of the methods disclosed herein, the composition is administered to the subject once a day. In embodiments of any of the methods disclosed herein, the composition is administered to the subject twice a day. In embodiments of any of the methods disclosed herein, the composition is administered to the subject three, four, five, six, seven, eight nine, ten times or more a day.

### Superoxide Dismutase/Soluble Fiber Compositions for Use in Methods

### 1) Superoxide Dismutase

The embodiments, the compositions disclosed herein comprise an amount of superoxide dismutase as measured in units of activity per mg of protein. For enzyme activity, one unit (U) (expressed in μmol/min) is defined as the amount of the enzyme that catalyzes the conversion of one micromole of substrate per minute under specified conditions. The units of superoxide dismutase activity can be measured by any known method. For example, methods of determining superoxide dismutase activity in units are described in McCord, J. M. and Fridovich, I., J. Biol. Chem. 1969, 244:6049-6055; Weydert et al., Nature Protocols 2010, 5 (1): 51-66; and in the technical at protocol https://www.sigmaaldrich.com/technical-documents/protocols/biology/enzymatic-assay-of-superoxide-dismutase.html; the disclosures of each of which are incorporated by reference herein. In embodiments, a unit of superoxide dismutase activity is defined as the amount of superoxide dismutase that will inhibit the rate of reduction of cytochrome c by 50% in a coupled system, using xanthine and xanthine oxidase at pH 7.8 at 25° C. in a 3.0 ml reaction volume.

In embodiments, the concentration of the amount of superoxide dismutase in the compositions disclosed herein is measured in the total amount of units of superoxide dismutase in the composition. In embodiments, the composition comprises from about 10 units to about 200 units superoxide dismutase. In embodiments, the composition comprises comprising from about 50 units to about 150 units superoxide dismutase. In embodiments, the composition comprises from about 70 units to about 100 units superoxide dismutase. In embodiments, the composition comprises from about 20 units to about 190 units superoxide dismutase. In embodiments, the composition comprises from about 30 units to about 180 units superoxide dismutase. In embodiments, the composition comprises from about 40 units to about 170 units superoxide dismutase. In embodiments, the composition comprises from about 50 units to about 160 units superoxide dismutase. In embodiments, the composition comprises from about 60 units to about 150 units superoxide dismutase. In embodiments, the composition comprises from about 70 units to about 140 units superoxide dismutase. In embodiments, the composition comprises from about 80 units to about 130 units superoxide dismutase. In embodiments, the composition comprises from about 90 units to about 130 units superoxide dismutase. In embodiments, the composition comprises from about 75 units to about 95 units superoxide dismutase. In embodiments, the composition comprises from about 80 units to about 90 units superoxide dismutase. In embodiments, the composition comprises about 20, 30, 40, 50, 60, 70, 80, 90, 100, 110, 120, 130, 140, 150, or 160 units superoxide dismutase. In embodiments, the composition comprises about 84 units superoxide dismutase.

The superoxide dismutase used in the compositions disclosed herein can be obtained from any source of the enzyme. In embodiments, the superoxide dismutase is extracted from melon, bovine liver, heterotrophic bacteria or marine phytoplankton. The superoxide dismutase can also be any type of the enzyme. In embodiments, the superoxide dismutase is a copper/zinc superoxide dismutase, an iron/manganese superoxide dismutase or a nickel superoxide dismutase.

In embodiments, the superoxide dismutase is extracted from a plant. In embodiments, the plant is a fruit, a grain or a tuber. In embodiments, the plant is a fruit selected from melon, citrus fruit, peach, pear, apple or banana. In embodiments, the plant is a grain selected from wheat, barley, rye, millet, oat, spelt, bulger, sorghum and farro. In embodiments, the plant is a tuber selected from horseradish, potato, yam, sweet potato, cassava or dahlia.

In embodiments, the superoxide dismutase is extracted from an animal. In embodiments, the animal is a cow, pig, sheep or goat.

In embodiments, the superoxide dismutase is extracted from a microorganism. In embodiments, the microorganism is phytoplankton or bacteria. In embodiments, the microorganism is a heterotrophic bacteria, e.g., a bacteria that takes the sugars it needs for energy production from their environment.

### 2) Soluble Fiber

The ratio of superoxide dismutase to soluble fiber can be adjusted as needed by varying the amount of either or both components of the composition. In embodiments, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

Without wishing to be bound by theory, the inventor has found that the presence of soluble fiber together with superoxide dismutase at specific ratios synergistically allows for improved stability and uptake in the digestive tract (gut) as demonstrated in the examples below. One mechanism behind this improved stability and uptake is that the soluble fiber forms a gel in the digestive tract and that this formed gel entraps and protects the superoxide dismutase while also allowing it to be more readily absorbed through the wall of the gut. The soluble fiber and the antioxidants formulations of the present disclosure provide synergistic effects in that the soluble prebiotic fiber acts as a fertilizer for the bacteria in the colon, while the polyphenols are absorbed in a substantial part in the colon and subject to extensive catabolism by colonic microbiota. The presence of the prebiotic fiber enhances the action of the colonic microbiota, thereby leading to more effective absorption of the polyphenols in the colon. A large majority of polyphenols from an antioxidant supplement end up in the large intestine where they undergo microbial metabolism into their active metabolites where they can exert an antioxidant effect. The soluble fiber modulates the gut microbes and maximizes polyphenol metabolism, producing many antioxidant, anti-inflammatory, and anti-infection effects.

In embodiments, the soluble fiber is a prebiotic fiber. As used herein a “prebiotic fiber” is a soluble fiber that forms a matrix in the digestive tract that provides a substrate for the propagation of microorganisms in the gut.

In embodiments of the compositions disclosed herein, various types of soluble fibers can be used, including mixtures of two, three, four, five, six or more different types of soluble fibers. In embodiments, the soluble fiber is a water soluble polysaccharide. In embodiments, the soluble fiber is isolated from corn, wheat, barley, rye, beans, apples, pears, peaches, citrus fruits, berries, peas, rice bran or oats. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble fiber is digestion-resistant maltodextrin. In embodiments, the soluble fiber is Fibersol-2® as sold by Archer Daniels Midland Company and Matsutani Chemical Industry Co., Ltd. In embodiments, the soluble fiber an alternative type of Fibersol-2® such as Fibersol-2AG, Fibersol-LQ, Fibersol-2L, Fibersol-DLQ or non-GMO Fibersol.

In some embodiments, the soluble fiber is corn-based digestion resistant maltodextrin (Fibersol-2) prebiotic fiber.

### 3) Gel Compositions

In embodiments of the compositions disclosed herein, the composition is in the form of a gel. In embodiments where the composition is in the form of a gel, it can be consumed in directly in that form. In other embodiments, the gel can be added to a beverage or food. In embodiments, the gel is mixed into water, a hydration beverage, a protein shake, juice, tea, coffee, milk, kefir, ice cream, yogurt, a smoothie, broth or soup prior to consumption.

In embodiments, the gel composition comprises from about 0.005 mg/mL to about 5.0 mg/mL superoxide dismutase. In embodiments, the gel composition comprises from about 0.01 mg/mL to about 2.5 mg/mL superoxide dismutase. In embodiments, the gel composition comprises from about 0.05 mg/mL to about 1.0 mg/mL superoxide dismutase. In embodiments, the gel composition comprises from about 0.1 mg/mL to about 0.5 mg/mL superoxide dismutase. In embodiments, the gel composition comprises about 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mg/mL superoxide dismutase.

In embodiments, the gel composition comprises from about 50 mg/mL to about 1000 mg/mL soluble fiber. In embodiments, the gel composition comprises from about 70 mg/mL to about 500 mg/mL soluble fiber. In embodiments, the gel composition comprises from about 90 mg/mL to about 250 mg/mL soluble fiber. In embodiments, the gel composition comprises from about 100 mg/mL to about 200 mg/mL soluble fiber. In embodiments, the gel composition comprises about 50, 75, 100, 110, 120, 125, 130, 133, 135, 140, 150, 160, 170, 175, 180, 190, 200, 225, 250, 275, 300, 325, 350, 375, 400, 425, 450, 475 or 500 mg/mL soluble fiber.

In embodiments of the compositions disclosed herein, the composition comprising superoxide dismutase and soluble fiber also comprises a fruit juice. The fruit juice can provide additional antioxidants, soluble fiber, insoluble fiber, vitamins and nutrients to the composition. In embodiments, the gel compositions disclosed herein comprise a fruit juice.

In embodiments, the fruit juice is pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice, prune juice or combinations thereof. In embodiments, the composition comprises two, three four, five or six types of juice selected from: pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice and prune juice.

In embodiments, the fruit juice can be concentrated, e.g., having some of the water in the original juice removed. In embodiments, the fruit juice is concentrated pomegranate juice, red grape juice, blueberry juice, dark sweet cherry juice, sour cherry juice, goji berry juice, acai berry juice, blackberry juice, raspberry juice, strawberry juice, gooseberry juice, cranberry juice, orange juice, grapefruit juice, watermelon juice, beet juice, apple juice, lemon juice, lime juice, lychee juice, pineapple juice, prune juice or combinations thereof. In embodiments, the composition comprises two, three four, five or six types of concentrated juice selected from: concentrated pomegranate juice, concentrated red grape juice, concentrated blueberry juice, concentrated dark sweet cherry juice, concentrated sour cherry juice, concentrated goji berry juice, concentrated acai berry juice, concentrated blackberry juice, concentrated raspberry juice, concentrated strawberry juice, concentrated gooseberry juice, concentrated cranberry juice, concentrated orange juice, concentrated grapefruit juice, concentrated watermelon juice, concentrated beet juice, concentrated apple juice, concentrated lemon juice, concentrated lime juice, concentrated lychee juice, concentrated pineapple juice, concentrated prune juice. In embodiments, the concentrated fruit juice has about 60% to about 97% of the water of the juice removed. In embodiments, the concentrated fruit juice has about 85% to about 95% of the water of the juice removed. In embodiments, the concentrated fruit juice has about 60%, 65%, 70%, 75%, 80%, 85%, 90%, 95%, 96%, 97%, 98% or 99% of the water of the fruit juice removed. Water can be removed from the fruit juices using any method known in the art for concentrating fruit juices.

In embodiments, the composition comprises from about 5 mg/mL to about 200 mg/ml of the concentrated fruit juice. In embodiments, the composition comprises from about 75 mg/mL to about 150 mg/mL concentrated pomegranate juice. In embodiments, the composition comprises from about 75 mg/mL to about 150 mg/mL concentrated red grape juice. In embodiments, the composition comprises from about 25 mg/mL to about 100 mg/ml concentrated blueberry juice. In embodiments, the composition comprises from about 20 mg/mL to about 80 mg/mL concentrated dark sweet cherry juice. In embodiments, the composition comprises from about 20 mg/mL to about 80 mg/mL concentrated sour cherry juice. In embodiments, the composition comprises from about 2 mg/mL to about 20 mg/mL concentrated goji berry juice. In embodiments, the composition comprises from about 2 mg/mL to about 20 mg/mL concentrated acai berry juice.

In embodiments of the gel compositions disclosed herein, the composition comprising superoxide dismutase and soluble fiber also comprises aloe vera. The aloe vera can provide additional antioxidants, soluble fiber, insoluble fiber, vitamins and nutrients to the composition.

In some embodiments, the aloe vera is concentrated aloe vera. In embodiments, the aloe vera can be concentrated, e.g., having some of the water removed from the aloe vera that is extracted from the aloe vera plant. In embodiments, the concentrated aloe vera has about 60% to about 97% of the water of the aloe vera removed. In embodiments, the concentrated aloe vera has about 60%, 65%, 70%, 75%, 80%, 85%, 90%, 95%, 96%, 97%, 98% or 99% of the water of the aloe vera removed. In embodiments, the composition comprises from about 2 mg/mL to about 20 mg/mL concentrated aloe vera.

In embodiments of the gel compositions disclosed herein, the composition comprising superoxide dismutase and soluble fiber also comprises green tea. The green tea can provide additional antioxidants, vitamins and nutrients to the composition.

In embodiments, the green tea is concentrated green tea. In embodiments, the concentrated green tea is a green tea extract made from green tea leaves or green tea powder. In embodiments, the green tea is added to the composition in powder form. In embodiments, the composition comprises from about 2 mg/mL to about 20 mg/mL concentrated green tea.

In embodiments of the gel compositions disclosed herein, the composition comprising superoxide dismutase and soluble fiber also comprises resveratrol. The resveratrol can provide additional antioxidants to the composition. In some embodiments, the composition comprises from about 0.5 mg/mL to about 6 mg/mL resveratrol. In embodiments, the composition comprises from about 1.5 mg/mL to about 3.0 mg/mL resveratrol. In embodiments, the composition comprises from about 2.0 mg/mL to about 2.7 mg/mL resveratrol. In embodiments, the composition comprises about 2.0 mg/mL resveratrol. In embodiments, the composition comprises about 2.67 mg/mL resveratrol.

In embodiments, a specific gel composition is provided in Table 1.

TABLE 1

| | | QTY |
|---|---|---|
| SI. # | Ingredient | in mg |
| 1 | Soluble corn fiber (Fibersol-2) | 4000 |
| 2 | Pomegranate Juice Concentrate | 4000 |
| 3 | Concord Grape Juice Concentrate | 4000 |
| 4 | Blueberry Juice Concentrate | 2000 |
| 5 | Dark Sweet Cherry Juice Concentrate | 1450 |
| 6 | Sour Cherry Juice Concentrate | 1450 |
| 7 | Goji Pulp | 294 |
| 8 | Acai Concentrate Juice | 200 |
| 9 | Aloe vera juice | 200 |
| 10 | Green Tea | 200 |
| 11 | Resveratrol | 80 |
| 12 | Extramel SOD (14000 unit/g) | 6 |
| 13 | CMC | 60.5 |
| 14 | Xanthan gum | 60.5 |
| 15 | QS Water (30 ml) | |

In embodiments, the gel composition is REVIVIFY® Pro-Vitality Gel.

### 4) Liquid Compositions

In embodiments, the present disclosure provides a liquid composition comprising superoxide dismutase, soluble fiber, and water. In embodiments, the present disclosure provides a liquid composition comprising: a) from about 0.03 units/mL to about 0.5 units/mL superoxide dismutase; b) from about 1.3 mg/mL to about 23 mg/mL soluble fiber; and c) water.

In embodiments, the liquid composition comprises from about 0.05 units/mL to about 0.4 units/mL superoxide dismutase. In embodiments, the liquid composition comprises from about 0.2 units/mL to about 0.3 units/mL superoxide dismutase. In embodiments, the liquid composition comprises from about 0.1 units/mL to about 0.4 units/mL superoxide dismutase. In embodiments, the liquid composition comprises from about 0.15 units/mL to about 0.35 units/mL superoxide dismutase. In embodiments, the liquid composition comprises about 0.05, 0.1, 0.15, 0.2, 0.25, 0.3, 0.35, or 0.4 units/mL superoxide dismutase. In embodiments, the liquid composition comprises about 0.20, 0.21, 0.22, 0.23, 0.24, 0.25, 0.26, 0.27, 0.28, 0.29 or 0.30 units/mL superoxide dismutase.

In embodiments, the liquid composition comprises from about 2.7 mg/mL to about 12 mg/mL soluble fiber. In embodiments, the liquid composition comprises from about 5.55 mg/mL to about 11.11 mg/mL soluble fiber. In embodiments, the liquid composition comprises from about 2 mg/mL to about 15 mg/mL soluble fiber. In embodiments, the liquid composition comprises from about 4 mg/mL to about 12 mg/mL soluble fiber. In embodiments, the liquid composition comprises from about 5 mg/mL to about 12 mg/mL soluble fiber. In embodiments, the liquid composition comprises from about 6 mg/mL to about 12 mg/mL soluble fiber. In embodiments, the liquid composition comprises from about 7 mg/mL to about 12 mg/mL soluble fiber. In embodiments, the liquid composition comprises from about 8 mg/mL to about 12 mg/mL soluble fiber. In embodiments, the liquid composition comprises from about 9 mg/mL to about 12 mg/mL soluble fiber. In embodiments, the liquid composition comprises from about 10 mg/mL to about 12 mg/mL soluble fiber. In embodiments, the liquid composition comprises about 2.78, 5.56, 8.33, 11.11 or 13.89 mg/mL soluble fiber.

In embodiments, the liquid composition comprises any of the superoxide dismutases disclosed herein. In embodiments, the superoxide dismutase is extracted from melon, bovine liver, heterotrophic bacteria or marine phytoplankton. In embodiments, the superoxide dismutase is a copper/zinc superoxide dismutase, an iron/manganese superoxide dismutase or a nickel superoxide dismutase.

In embodiments of the liquid composition, the ratio of superoxide dismutase to soluble fiber in the composition is from about 1:100 to about 1:1000 by weight. In embodiments, the ratio of superoxide dismutase to soluble fiber in the composition is from about 1:200 to about 1:800 by weight. In embodiments, the ratio of superoxide dismutase to soluble fiber in the composition is from about 1:300 to about 1:700 by weight. In embodiments, the ratio of superoxide dismutase to soluble fiber in the composition is from about 1:500 to about 1:700 by weight. In embodiments, the ratio of superoxide dismutase to soluble fiber in the composition is from about 1:650 to 1:675 by weight.

In embodiments, the liquid composition comprises any of the soluble fibers disclosed herein. In embodiments, the soluble fiber is a prebiotic fiber. In embodiments, the soluble fiber is a water soluble polysaccharide. In embodiments, the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides. In embodiments, the soluble fiber is soluble corn fiber. In embodiments, the soluble corn fiber is digestion-resistant maltodextrin.

In embodiments, the liquid compositions can further comprise additional ingredients, including sweeteners, pH adjusting agents, flavors and other agents, including combinations of these agents.

In embodiments, the liquid composition comprises a sweetener. In embodiments, the liquid composition comprises a combination of sweeteners, examples of which are provided below.

In embodiments, the sweetener is a simple sugar. In embodiments, the simple sugar is ribose, glucose, fructose, sucrose, lactose or combinations thereof. In embodiments, the simple sugar is ribose. In embodiments, the simple sugar is d-ribose.

In embodiments, the liquid composition comprises from about 0.1 mg/mL to about 1.5 mg/mL of a simple sugar. In embodiments, the liquid composition comprises from about 0.2 mg/mL to about 1.3 mg/mL of a simple sugar. In embodiments, the liquid composition comprises from about 0.3 mg/mL to about 1.0 mg/mL of a simple sugar. In embodiments, the liquid composition comprises from about 0.4 mg/mL to about 0.8 mg/mL of a simple sugar. In embodiments, the liquid composition comprises from about 0.4 mg/mL to about 0.85 mg/ml of a simple sugar. In embodiments, the liquid composition comprises from about 0.4 mg/mL to about 0.6 mg/mL of a simple sugar. In embodiments, the liquid composition comprises from about 0.4, 0.45, 0.5, 0.55, 0.6, 0.65, 0.7, 0.75, 0.8, or 0.85 mg/mL of a simple sugar.

In embodiments, the liquid composition comprises from about 0.1 mg/mL to about 1.5 mg/mL of d-ribose. In embodiments, the liquid composition comprises from about 0.2 mg/mL to about 1.3 mg/mL of d-ribose. In embodiments, the liquid composition comprises from about 0.3 mg/mL to about 1.0 mg/mL of d-ribose. In embodiments, the liquid composition comprises from about 0.4 mg/mL to about 0.8 mg/mL of d-ribose. In embodiments, the liquid composition comprises from about 0.4 mg/mL to about 0.85 mg/mL of d-ribose. In embodiments, the liquid composition comprises from about 0.4 mg/mL to about 0.6 mg/mL of d-ribose. In embodiments, the liquid composition comprises from about 0.4, 0.45, 0.5, 0.55, 0.6, 0.65, 0.7, 0.75, 0.8, or 0.85 mg/mL of d-ribose.

In embodiments of the liquid composition, the sweetener comprises a sugar alcohol. In embodiments, the sugar alcohol is erythritol, mannitol, sorbitol, xylitol, lactitol, isomalt or combinations thereof. In embodiments, the sugar alcohol is erythritol.

In embodiments, the liquid composition comprises from about 1.3 mg/mL to about 9.0 mg/mL of a sugar alcohol. In embodiments, the liquid composition comprises from about 2.0 mg/mL to about 8.0 mg/mL of a sugar alcohol. In embodiments, the liquid composition comprises from about 3.9 mg/mL to about 7.0 mg/mL of a sugar alcohol. In embodiments, the liquid composition comprises from 2.7 mg/mL to about 5.6 mg/mL of a sugar alcohol. In embodiments, the liquid composition comprises about 2.0, 2.5, 3.0, 3.5, 4.0, 4.15, 4.5, 5.0, 5.5, 6.0, 6.5, 7.0, 7.5 or 8.0 mg/mL of a sugar alcohol.

In embodiments, the liquid composition comprises from about 1.3 mg/mL to about 9.0 mg/mL of erythritol. In embodiments, the liquid composition comprises from about 2.0 mg/ml to about 8.0 mg/mL of erythritol. In embodiments, the liquid composition comprises from about 3.9 mg/mL to about 7.0 mg/mL of erythritol. In embodiments, the liquid composition comprises from 2.7 mg/mL to about 5.6 mg/mL of erythritol. In embodiments, the liquid composition comprises about 2.0, 2.5, 3.0, 3.5, 4.0, 4.15, 4.5, 5.0, 5.5, 6.0, 6.5, 7.0, 7.5 or 8.0 mg/mL of erythritol.

In embodiments, the sweetener is a steviol glycoside. In embodiments, the steviol glycoside is Rebaudioside A, Stevioside, Rebaudioside C, Dulcoside A, Rebaudioside B, Rebaudioside D, Rebaudioside E, steviolbioside or combinations thereof.

In embodiments, the liquid composition comprises from about 0.05 mg/mL to about 0.75 mg/mL of steviol glycoside. In embodiments, the liquid composition comprises from about 0.2 mg/mL to about 0.35 mg/mL steviol glycoside. In embodiments, the liquid composition comprises from about 0.1 mg/mL to about 0.5 mg/mL steviol glycoside. In embodiments, the liquid composition comprises from about 0.2 mg/mL to about 0.3 mg/mL steviol glycoside. In embodiments, the liquid composition comprises about 0.10, 0.15, 0.20, 0.25, 0.27, 0.30, 0.35, 0.40, 0.45 or 0.50 mg/mL steviol glycoside.

In embodiments, the liquid composition comprises from about 0.05 mg/mL to about 0.75 mg/mL of Rebaudioside A. In embodiments, the liquid composition comprises from about 0.2 mg/mL to about 0.35 mg/mL Rebaudioside A. In embodiments, the liquid composition comprises from about 0.1 mg/mL to about 0.5 mg/mL Rebaudioside A. In embodiments, the liquid composition comprises from about 0.2 mg/mL to about 0.3 mg/mL Rebaudioside A. In embodiments, the liquid composition comprises about 0.10, 0.15, 0.20, 0.25, 0.27, 0.30, 0.35, 0.40, 0.45 or 0.50 mg/mL Rebaudioside A.

In embodiments, the liquid composition further comprises a pH adjusting agent. In embodiments, the pH adjusting agent is any food-safe agent that can be used to change the pH of the liquid composition. In embodiments, the pH adjusting agent is citric acid, acetic acid, hydrochloric acid, lactic acid, malic acid, phosphoric acid, tartaric acid or combinations thereof.

In embodiments, the liquid composition comprises from about 0.1 mg/mL to about 1.5 mg/mL of a pH adjusting agent. In embodiments, the liquid composition comprises from about 0.2 mg/mL to about 1.3 mg/mL of a pH adjusting agent. In embodiments, the liquid composition comprises from about 0.3 mg/mL to about 1.0 mg/mL of a pH adjusting agent. In embodiments, the liquid composition comprises from about 0.4 mg/mL to about 0.8 mg/mL of a pH adjusting agent. In embodiments, the liquid composition comprises from about 0.4 mg/mL to about 0.85 mg/mL of a pH adjusting agent. In embodiments, the liquid composition comprises from about 0.4 mg/mL to about 0.6 mg/mL of a pH adjusting agent. In embodiments, the liquid composition comprises from about 0.4, 0.45, 0.5, 0.55, 0.6, 0.65, 0.7, 0.75, 0.8, or 0.85 mg/mL of a pH adjusting agent.

In embodiments, the liquid composition comprises from about 0.1 mg/mL to about 1.5 mg/mL of citric acid. In embodiments, the liquid composition comprises from about 0.2 mg/ml to about 1.3 mg/mL of citric acid. In embodiments, the liquid composition comprises from about 0.3 mg/mL to about 1.0 mg/mL of citric acid. In embodiments, the liquid composition comprises from about 0.4 mg/mL to about 0.8 mg/mL of citric acid. In embodiments, the liquid composition comprises from about 0.4 mg/mL to about 0.85 mg/mL of citric acid. In embodiments, the liquid composition comprises from about 0.4 mg/mL to about 0.6 mg/ml of citric acid. In embodiments, the liquid composition comprises from about 0.4, 0.45, 0.5, 0.55, 0.6, 0.65, 0.7, 0.75, 0.8, or 0.85 mg/mL of citric acid.

In embodiments, the liquid composition comprises a flavoring. In embodiments, the flavor is a natural flavoring. In embodiments, the flavor is an artificial flavoring. In embodiments, the flavor if a fruit juice flavoring. In embodiments, the flavoring is pomegranate, red grape, blueberry, dark sweet cherry, sour cherry, goji berry, acai berry, blackberry, raspberry, strawberry, gooseberry, cranberry, orange, grapefruit, watermelon, beet, apple, lemon, lime, lychee, pineapple, prune, mango or combinations thereof. In embodiments, the liquid composition comprises a cola flavoring.

In embodiments, the liquid composition is formulated in a beverage. In embodiments, the liquid composition is formulated as a hydration beverage, a protein shake, juice, tea, coffee, milk, kefir, ice cream, yogurt, a smoothie, broth or soup.

In embodiments where the compositions are formulated into a beverage, an antifoaming agent can be added during manufacturing. In embodiments, the antifoaming agent used in simethicone. In embodiments, the antifoaming agent used is a fatty alcohol (such as cetostearyl alcohol), an insoluble oil (such as castor oil), a stearate, a polydimethylsiloxane and other silicones derivatives, ethers and glycols, or mixtures thereof.

In embodiments, the liquid composition has a volume of from about 15 mL to about 1500 mL. In embodiments, the liquid composition has a volume of from about 30 mL to about 1200 mL. In embodiments, the liquid composition has a volume of from about 50 mL to about 1000 mL. In embodiments, the liquid composition has a volume of from about 100 mL to about 500 mL. In embodiments, the liquid composition has a volume of from about 200 mL to about 400 mL. In embodiments, the liquid composition has a volume of from about 200 mL to about 1000 mL. In embodiments, the liquid composition has a volume of from about 300 mL to about 1000 mL. In embodiments, the liquid composition has a volume of about 50, 100, 150, 200, 250, 300, 350, 400, 450, 500, 550, 600, 650, 700, 750, 800, 850, 900, 950 or 1000 mL.

In embodiments, the liquid composition is packaged in a bottle. In embodiments, the bottle is a glass bottle. In embodiments, the bottle is a plastic bottle. In embodiments, the liquid composition is packaged in a can. In embodiments, the liquid composition is packaged in a drink box.

In embodiments, a specific example of a liquid composition is provided in Table 2:

TABLE 2

| 1. | SOD [melon extract powder, | 6.0 | mg |
|---|---|---|---|
| | 14,000 units/g] | [84 enzyme units] | |
| 2. | Fibersol-2 | 4000.0 | mg |
| 3. | D-Ribose | 200.0 | mg |
| 4. | Erythritol | 1494.0 | mg |
| 5. | Citric acid | 200.0 | mg |
| 6. | Rebaudioside A | 100.0 | mg |
| 7. | Flavor | 100.0 | mg |
| 8. | Water qs | 360 | ml. |

### 5) Powder Compositions

In some embodiments of the compositions disclosed herein, the composition is in the form of a powder. In embodiments where the composition is in the form of a powder, it can be consumed in dry powder form or added to a beverage or food. In embodiments, the powder is mixed into water, a hydration beverage, a protein shake, juice, tea, coffee, milk, kefir, ice cream, yogurt, a smoothie, broth or soup prior to consumption.

In embodiments, the powder composition comprises a high concentration of superoxide dismutase by weight, e.g., greater than 0.1 ppm, greater than 0.5 ppm, greater than 1 ppm, greater than 2 ppm, greater than 5 ppm, greater than 10 ppm, greater than 20 ppm, greater than 50 ppm, greater than 100 ppm, greater than 200 ppm, greater than 300 ppm, greater than 400 ppm or greater than 500 ppm. In embodiments, the powder composition comprises from about 0.1 ppm to about 10 ppm superoxide dismutase by weight. In embodiments, the powder composition comprises from about 0.5 ppm to about 5 ppm superoxide dismutase by weight. In embodiments, the powder composition comprises from about 0.7 ppm to about 2 ppm superoxide dismutase by weight. In embodiments, the powder composition comprises from about 0.8 ppm to about 1.2 ppm superoxide dismutase by weight. In embodiments, the powder composition comprises about 0.1, 0.25, 0.5, 0.75, 1.0, 1.1, 1.2, 1.25, 1.3, 1.4, 1.5, 1.6, 1.7, 1.75, 1.8, 1.9, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, or 10.0 ppm superoxide dismutase by weight.

In embodiments, the powder composition comprises from about 5 ppm to about 15 ppm superoxide dismutase by weight. In embodiments, the powder composition comprises from about 7.5 ppm to about 12.5 ppm superoxide dismutase by weight. In embodiments, the powder composition comprises from about 8 ppm to about 11 ppm superoxide dismutase by weight. In embodiments, the powder composition comprises from about 9 ppm to about 11 ppm superoxide dismutase by weight. In embodiments, the powder composition comprises about 5, 6, 7, 8, 9, 10, 11, 12, 13, 14 or 15 ppm superoxide dismutase by weight.

In embodiments, the powder composition comprises from about 1% to about 90% soluble fiber by weight. In embodiments, the powder composition comprises from about 1% to about 50% soluble fiber by weight. In embodiments, the powder composition comprises from about 5% to about 25% soluble fiber by weight. In embodiments, the powder composition comprises from about 10% to about 20% soluble fiber by weight. In embodiments, the powder composition comprises from about 12% to about 14% soluble fiber by weight. In embodiments, the power composition comprises about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% soluble fiber by weight.

In embodiments, the powder composition comprises from about 50% to about 75% soluble fiber by weight. In embodiments, the powder composition comprises from about 50% to about 70% soluble fiber by weight. In embodiments, the powder composition comprises from about 60% to about 70% soluble fiber by weight. In embodiments, the powder composition comprises from about 50% to about 80% soluble fiber by weight. In embodiments, the power composition comprises about 50%, 55%, 60%, 65%, 66%, 67%, 70%, 75%, 80%, 85% or 90% soluble fiber by weight.

In embodiments, the powder composition comprises a sweetener. In embodiments, the powder composition comprises a combination of sweeteners, examples of which are provided below.

In embodiments, the sweetener is a simple sugar. In embodiments, the simple sugar is ribose, glucose, fructose, sucrose, lactose or combinations thereof. In embodiments, the simple sugar is ribose. In embodiments, the simple sugar is d-ribose.

In embodiments, the powder composition comprises from about 1% to about 10% by weight of a simple sugar. In embodiments, the powder composition comprises from about 2% to about 8% by weight of a simple sugar. In embodiments, the powder composition comprises from about 2% to about 5% by weight of a simple sugar. In embodiments, the powder composition comprises from about 1%, 2%, 3%, 4%, 5%, 6%, 7%, 8%, 9% or 10% by weight of a simple sugar.

In embodiments, the powder composition comprises from about 1% to about 10% by weight of d-ribose. In embodiments, the powder composition comprises from about 2% to about 8% by weight of d-ribose. In embodiments, the powder composition comprises from about 2% to about 5% by weight of d-ribose. In embodiments, the powder composition comprises from about 1%, 2%, 3%, 4%, 5%, 6%, 7%, 8%, 9% or 10% by weight of d-ribose.

In embodiments of the powder composition, the sweetener comprises a sugar alcohol. In embodiments, the sugar alcohol is erythritol, mannitol, sorbitol, xylitol, lactitol, isomalt or combinations thereof.

In embodiments, the powder composition comprises from about 10% to about 50% by weight of erythritol. In embodiments, the powder composition comprises from about 15% to about 35% by weight of erythritol. In embodiments, the powder composition comprises from about 20% to about 30% by weight of erythritol. In embodiments, the powder composition comprises from about 15%, 20%, 25, 30%, 35%, 40%, 45% or 50% by weight of erythritol.

In embodiments, the sweetener is a steviol glycoside. In embodiments, the steviol glycoside is Rebaudioside A, Stevioside, Rebaudioside C, Dulcoside A, Rebaudioside B, Rebaudioside D, Rebaudioside E, steviolbioside or combinations thereof.

In embodiments, the powder composition comprises from about 0.5% to about 5% by weight of a steviol glycoside. In embodiments, the powder composition comprises from about 1% to about 4% by weight of a steviol glycoside. In embodiments, the powder composition comprises from about 1% to about 2.5% by weight of a steviol glycoside. In embodiments, the powder composition comprises from about 1%, 1.5%, 1.6% 2%, 2.5%, 3%, 3.5%, or 4% by weight of a steviol glycoside.

In embodiments, the powder composition comprises from about 0.5% to about 5% by weight of Rebaudioside A. In embodiments, the powder composition comprises from about 1% to about 4% by weight of Rebaudioside A. In embodiments, the powder composition comprises from about 1% to about 2.5% by weight of Rebaudioside A. In embodiments, the powder composition comprises from about 1%, 1.5%, 1.6% 2%, 2.5%, 3%, 3.5%, or 4% by weight of Rebaudioside A.

In embodiments, the liquid composition further comprises a pH adjusting agent. In embodiments, the pH adjusting agent is any food-safe agent that can be used to change the pH of the liquid composition. In embodiments, the pH adjusting agent is citric acid, acetic acid, hydrochloric acid, lactic acid, malic acid, phosphoric acid, tartaric acid or combinations thereof.

In embodiments, the powder composition comprises from about 1% to about 10% by weight of a pH adjusting agent. In embodiments, the powder composition comprises from about 2% to about 8% by weight of a pH adjusting agent. In embodiments, the powder composition comprises from about 2% to about 5% by weight of a pH adjusting agent. In embodiments, the powder composition comprises from about 1%, 2%, 3%, 4%, 5%, 6%, 7%, 8%, 9% or 10% by weight of a pH adjusting agent.

In embodiments, the powder composition comprises from about 1% to about 10% by weight of citric acid. In embodiments, the powder composition comprises from about 2% to about 8% by weight of citric acid. In embodiments, the powder composition comprises from about 2% to about 5% by weight of citric acid. In embodiments, the powder composition comprises from about 1%, 2%, 3%, 4%, 5%, 6%, 7%, 8%, 9% or 10% by weight of citric acid.

In embodiments, a specific example of a powder composition is provided in Table 3:

TABLE 3
Total amount: 6 grams.

| 1. | SOD [melon extract powder, | 6.0 | mg |
|---|---|---|---|
| | 14,000 units/g] | [84 enzyme units] | |
| 2. | Fibersol-2 | 4000.0 | mg |
| 3. | D-Ribose | 200.0 | mg |
| 4. | Erythritol [excipients] | 1494.0 | mg |
| 5. | Citric acid [excipients] | 200.0 | mg |
| 6. | Sweetener [Rebaudioside A], | 100.0 | mg |
| | natural stevia | | |

### 6) Excipients/Formulations

In embodiments, the compositions disclosed herein can be combined with one or more excipient. In embodiments, the excipient is a gelling agent, a thickening agent, a carrier, a buffer or a filler. In embodiments, the compositions disclosed herein can be formulated into beverages or foodstuffs. In embodiments, the compositions are formulated into juice, a hydration beverage (e.g. a sports drink), a protein shake, tea, coffee, milk, kefir, ice cream, yogurt, a smoothie, broth or soup.

In embodiments where the compositions are formulated into a beverage, an antifoaming agent can be added during manufacturing. In embodiments, the antifoaming agent used in simethicone. In embodiments, the antifoaming agent used is a fatty alcohol (such as cetostearyl alcohol), an insoluble oil (such as castor oil), a stearate, a polydimethylsiloxane and other silicones derivatives, ethers and glycols, or mixtures thereof.

In embodiments, the compositions comprise the thickener/gelling agent carboxymethyl cellulose. In embodiments, composition is a gel comprising superoxide dismutase, soluble fiber and carboxymethyl cellulose. In embodiments, the composition comprises from about 0.5 to about 5.0 mg/mL carboxymethyl cellulose.

In embodiments, the compositions comprise the thickener/gelling agent xanthan gum. In embodiments, composition is a gel comprising superoxide dismutase, soluble fiber and xanthan gum. In embodiments, the composition comprises from about 0.5 to about 5.0 mg/ml xanthan gum.

### EXAMPLES

### Example 1: SOD and Soluble Fiber Gel Attenuates Human Brain Microvascular Endothelial Cells (HBMEC) from Hypoxia Induced Disruption of the Blood Brain Barrier (BBB) in an In Vitro Model

### Background and Objective

Accumulating data suggests that oxidative stress and mitochondrial damage are involved in the pathogenesis of neurodegenerative disorders including Parkinson Disease (PD), Multiple Sclerosis (MS), Alzheimer's Disease (AD), and many others. The brain uses about 20% of the body's total oxygen consumption, and thus is a high producer of reactive oxygen species (ROS). Also, the brain cell membrane composed of more unsaturated fatty acids (monounsaturated fatty acids (MUFAs) and polyunsaturated fatty acids (PUFAs)), and thus is more prone to lipid auto-oxidation due to ROS. REVIVIFY™ superoxide dismutase (SOD) gel provides an instant reduction of oxidative stress from multi-dimensional pathways and resulted in an immediate effect induced by the disease symptoms. The purpose of the study in this Example was to evaluate whether SOD gel attenuates human brain microvascular endothelial cells (HBMEC) from oxidative damage. The objective of the study was to evaluate whether SOD gel attenuates the hypoxia induced disruption of HBMEC Monolayer Permeability.

REVIVIFY™ pro-vitality antioxidant gel is composed of primary antioxidant superoxide dismutase (SOD), prebiotic fibers, and diverse polyphenols from various fruit juices. REVIVIFY™ gel has a formulation as shown in Table 4.

TABLE 4

| | | QTY |
|---|---|---|
| SI. # | Ingredient | in mg |
| 1 | Soluble corn fiber (Fibersol-2) | 4000 |
| 2 | Pomegranate Juice Concentrate | 4000 |
| 3 | Concord Grape Juice Concentrate | 4000 |
| 4 | Blueberry Juice Concentrate | 2000 |
| 5 | Dark Sweet Cherry Juice Concentrate | 1450 |
| 6 | Sour Cherry Juice Concentrate | 1450 |
| 7 | Goji Pulp | 294 |
| 8 | Acai Concentrate Juice | 200 |
| 9 | Aloe vera juice | 200 |
| 10 | Green Tea | 200 |
| 11 | Resveratrol | 80 |
| 12 | Extramel SOD (14000 unit/g) | 6 |
| 13 | CMC | 60.5 |
| 14 | Xanthan | 60.5 |
| 15 | QS Water (30 ml) | |

### Study Design

Human brain microvascular endothelial cells (HBMEC) were seeded on 6 well plates in hypoxia conditions (2% oxygen). Prior to treatment, cells were incubated in serum free media for 24 hours. Cells were treated with the following agents: 1. Superoxide Dismutase only; 2. Prebiotic fiber only; 3. Fruit juice only; 4. superoxide Dismutase+Prebiotic fiber+Fruit juice (Combination); 5. Negative Control. Treated cells were incubated in cell culture media for 48 hours.

The monolayer permeability study was performed by method previously described (Uddin, N. M. et al., Am J Physiol Regul Integr Comp Physiol 296: R1726-R1734, 2009) under hypoxia conditions and pretreatment with revivify. HBMEC were grown on poly-L-lysine glass chamber slides. The cells were treated with the treatment conditions mentioned above. After exposure to hypoxia condition, cells were washed in PBS and fixed in 4% paraformaldehyde.

After repeated washing steps, Triton X-100 treatment, and blocking for nonspecific binding, cells were incubated with a primary antibody for the cell tight junction proteins ZO-1 (Zonula occludens-1), Occludin, Claudin-1 or E-Cadherin (Invitrogen) at 4° C. overnight. Cells were washed in PBS and exposed to an FITC-conjugated secondary antibody for 1 h. After repeated washing steps, the cells were mounted in an antifade mounting medium that contained the nuclear stain DAPI (Invitrogen, Eugene, OR). Cells were observed under an Olympus FluoView FV 300 confocal laser-scanning microscope with appropriate filters for visualizing FITC and DAPI.

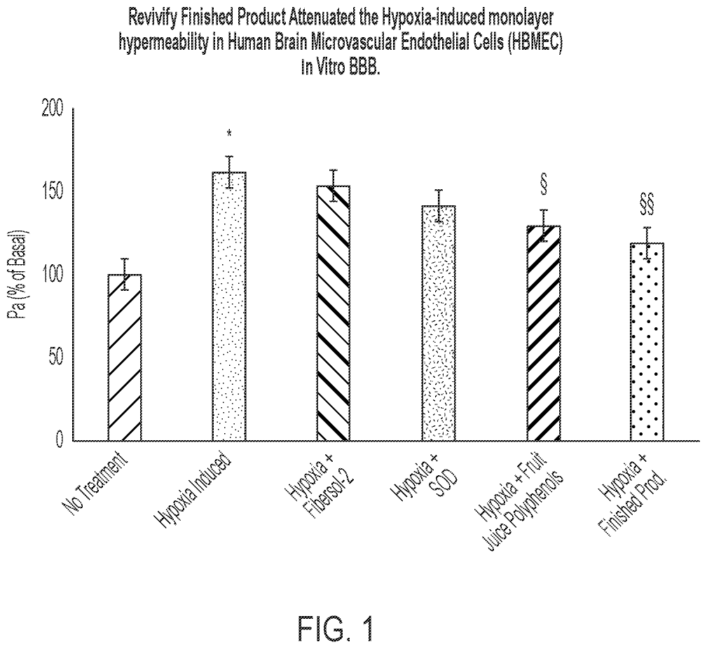

The results of the monolayer permeability study are plotted in FIG. 1. As can be seen in FIG. 1, while hypoxia exposure alone causes an increase in permeability compared to the basal level, hypoxia exposure along with the SOD gel (Revivify Finished Product) leads to a significant decrease in permeability.

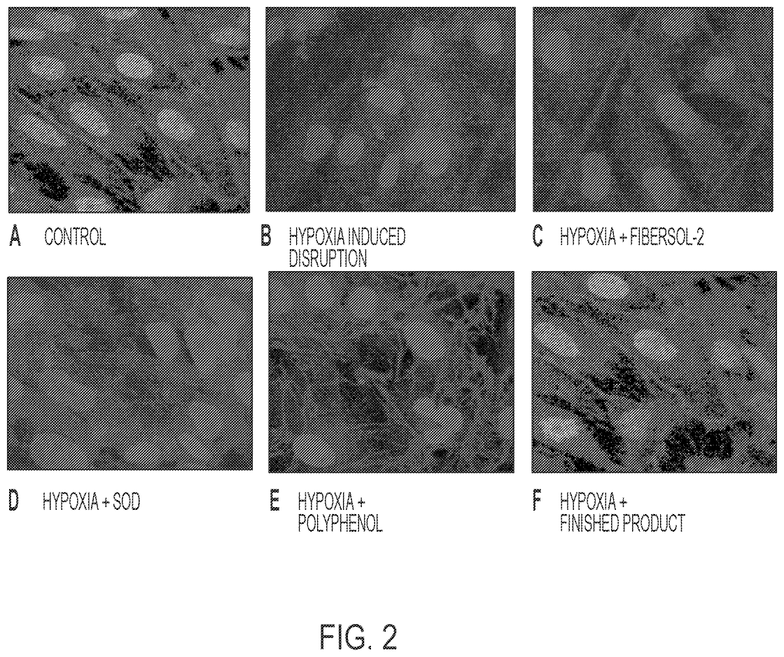

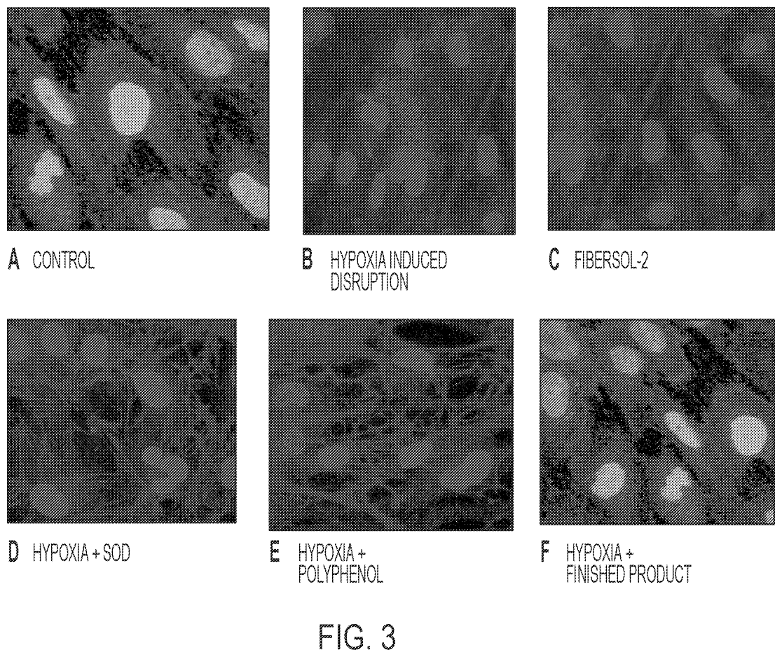

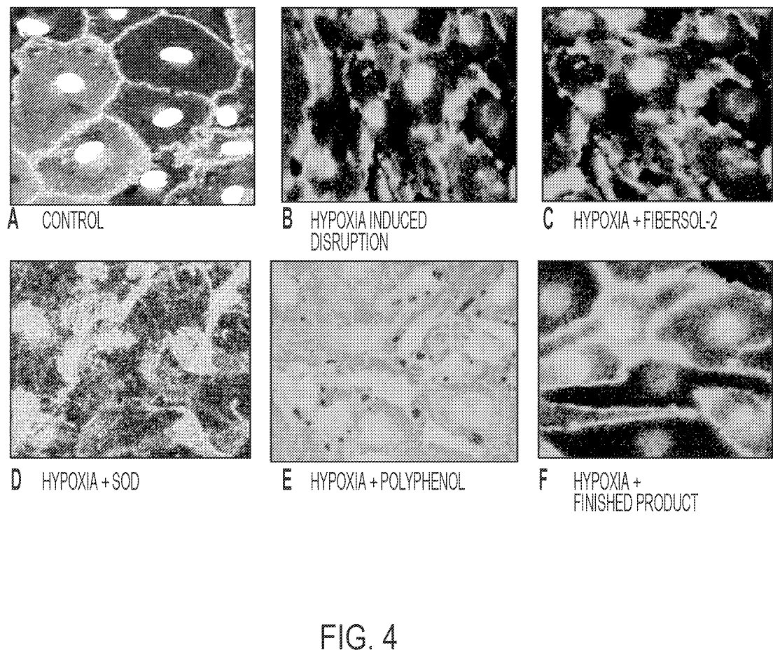

Cell imagining results are shown in FIG. 2 (ZO-1 detection), FIG. 3 (Occludin detection) and FIG. 4 (E-cadherin). As can be seen in the images, while treatment with SOD alone and polyphenols (fruit juices) alone protects some of the tight junction formations, treatment with finished product SOD gel results in maintenance of tight junctions that is very similar to untreated control (compare panels A and F in each figure). These results thus show that SOD gel is able to maintain the integrity of an HBMEC layer under hypoxic conditions.

### Results

This study demonstrates that:
HBMEC monolayer permeability was significantly increased under hypoxic conditions. SOD gel significantly attenuated the hyperpermeability induced by hypoxia.
SOD gel Finished Product attenuated the hypoxia-induced monolayer hyperpermeability in a Human Brain Microvascular Endothelial Cells (HBMEC) in vitro blood brain barrier model.
Immunofluorescence images of endothelial cell tight junction proteins: ZO-1, Occludin and E-Cadherin in HBMEC show that SOD gel maintained cell tight junctions under hypoxia conditions.
CONTROL (No treatment) cells show intact tight junctions evidenced by the strong and continuous presence of ZO-1, Occludin and E-Cadherin at the junctions. HYPOXIA exposed cells show disruption of the tight junction proteins. HYPOXIA+FIBERSOL exposed cells show no attenuation of tight junction proteins. HYPOXIA+SOD exposed cells show partial attenuation of tight junction proteins. HYPOXIA+POLYPHENOL exposed cells show attenuation of tight junction proteins. HYPOXIA+FINISHED PRODUCT exposed cells show attenuation of tight junction proteins.

### Conclusions

Pertaining to PD, SOD gel can improve motor activity, muscle stiffness, and overall body response with less exhaustion. For AD, it may improve the memory response, coordination with surrounding atmosphere. For other neurological disorders, it can improve focus, concentration, and alertness, which may be beneficial to people with learning disabilities, people with autistic spectrum disorders, people with mental exhaustion, and can benefit to the people who need study focus, or who work jobs requiring high concentration.

Pre-biotic soluble corn fiber in the SOD gel support a healthy gut eco-system where the modulation of beneficiary microbes influences various positive neurological effects. The gut-brain bi-directional axis can relate instant neuro-responses.

### Example 2: SOD and Soluble Fiber Gel Reduces Fasting Glucose: Human Case Study

### Background and Objective

REVIVIFY™ pro-vitality antioxidant gel is composed of the primary antioxidant superoxide dismutase (SOD), prebiotic fibers, diverse polyphenols from various fruit juices. SOD diminishes the superoxide anion that is produced due to normal cellular activity. Polyphenols are phenolic compounds act as antioxidant, anti-inflammatory, and anti-viral agents. SOD gel repairs damaged cells due to reactive oxygen molecules of ROS/RNS. Dietary prebiotic fibers modulate beneficiary gut eco microbiomes and provide many health benefits including immunity. The combination of these three components stimulates immunity via T-cell activation and antioxidative and anti-inflammatory pathways. Glucose metabolism with diabetic patient is known as a metabolic disorder. There is a belief that glucose metabolism disorder is due to oxidative stress of body cells particularly liver cells, pancreatic cells, kidney cells and brain cells, along with a negative impact from gut microbes.

The objective of the study in this Example was to evaluate whether revivify reduces the fasting glucose level in type 2 diabetic patients.

### Study Design

A case and control study was performed in a population from Williamson County, Texas. In total, 30 subjects participated in the study who were equally and randomly assigned to treatment groups. There were 18 males and 12 females. The selection criteria was: males and females aged 20-60 years with type to diabetes 2 diabetes. The age of each subject is provided in Table 7 below. The subjects had an average Fasting Blood Glucose (mmol/l) of 7.8±1.2 and HbA1c levels of 8.2±2.1.

Subjects used one pouch of REVIVIFY SOD gel daily after breakfast and one package of REVIVIFY SOD powder dissolved in water while continuing taking a combination of any two of the following oral diabetic medications daily for: metformin 500 mg; jardiance 10 mg; glipizide 10 mg; Lantus Insulin Glargine 30 units; and Novolong Insulin Aspart 15 units (twice daily). Subjects had been taking their combination of oral diabetic mediciations for at least one year. The SOD gel used was the formulation from Example 1. The powder is formulated as shown in Table 5 below. The drug combinations taken by each individual subject are shown in Table 7.

TABLE 5
Total amount: 6 grams.

| 1. | SOD [melon extract powder, | 6.0 | mg |
|---|---|---|---|
| | 14,000 units/g] | [84 enzyme units] | |
| 2. | Fibersol-2 | 4000.0 | mg |
| 3. | D-Ribose | 200.0 | mg |
| 4. | Erythritol [excipients] | 1494.0 | mg |
| 5. | Citric acid [excipients] | 200.0 | mg |
| 6. | Sweetener [Rebaudioside A], | 100.0 | mg |
| | natural stevia | | |

### Results

After three months of taking REVIVIFY gel and powder, a significant decrease of average Fasting Blood Glucose (mmol/l) and HbA1c was observed.

The Fasting Blood Glucose (mmol/l) was decreased from 7.8±1.2 (initial; beginning of study) to 5.4±0.4 (after three months of study) and HbA1c was decreased from 8.2±2.1 (initial; beginning of study) to 6.8±2.4 (after three months of study). A summary of the results is provided in Table 6 below. Results for individual subjects at the beginning and end of the study are provided in Table 7.

Moreover, the subjects reported an improvement of their general health, overall fatigue and sleeping patterns.

TABLE 6

| | Week | Week | Week | Week | Week | Week | Week | Week | Week | Week | Week | Week | Week |
|---|---|---|---|---|---|---|---|---|---|---|---|---|---|
| Time | 0 | 1 | 2 | 3 | 4 | 5 | 6 | 7 | 8 | 9 | 10 | 11 | 12 |
| Fasting | 7.8 ± | 7.6 ± | 7.2 ± | 6.9 ± | 6.9 ± | 6.8 ± | 6.8 ± | 6.5 ± | 5.8 ± | 5.8 ± | 5.5 ± | 5.3 ± | 5.1 ± |
| Blood | 1.2 | 1.6 | 1.5 | 1.0 | 0.8 | 1.1 | 1.3 | 0.8 | 0.9 | 1.1 | 0.6 | 0.9 | 0.1 |
| Glucose | | | | | | | | | | | | | |
| (mmol/L) | | | | | | | | | | | | | |
| HbA1C (%) | 8.2 ± | | | | | | | | | | | | 6.8 ± |
| | 2.1 | | | | | | | | | | | | 2.1 |
| General | Poor | Poor | Poor | Poor | Poor | Poor | Fair | Fair | Fair | Fair | Fair | Fair | Good |
| Health | | | | | | | | | | | | | |
| Fatigue | Fatigue | Fatigue | Fatigue | Fatigue | Fatigue | Fatigue | Less | Less | Less | Less | Less | Less | No |
| Status | | | | | | | Fatigue | Fatigue | Fatigue | Fatigue | Fatigue | Fatigue | Fatigue |
| Sleeping | Moder- | Moder- | Moder- | Moder- | Moder- | Fair | Fair | Fair | Fair | Fair | Fair | Good | Good |
| Pattern | ate | ate | ate | ate | ate | | | | | | | | |

TABLE 7

| | | | Week 0 | Week l2 | Week 0 | Week 12 |
|---|---|---|---|---|---|---|
| Subject | Age | Drugs | A1C | A1C | Fasting Glucose | Fasting Glucose |
| M1 | 41 | 1, 2 | 8.9 | 7.2 | 7.9 | 5.2 |
| M2 | 50 | 3, l | 8.3 | 6.8 | 8.5 | 5.4 |
| M3 | 42 | 1, 2 | 8.2 | 7.1 | 8.1 | 5.4 |
| M4 | 46 | 3, 1 | 8.8 | 7.5 | 7.8 | 5.3 |
| M5 | 46 | 3, l | 7.8 | 6.2 | 8.1 | 5.2 |
| M6 | 45 | 3, 1 | 8.4 | 7.1 | 7.5 | 5.2 |
| M7 | 47 | 3, 1 | 7.9 | 5.8 | 8.2 | 5.2 |
| M8 | 42 | 1, 2 | 8.5 | 7.1 | 7.5 | 5.4 |
| M9 | 42 | 1, 2 | 8.1 | 7.1 | 8.4 | 5.3 |
| M10 | 49 | 3, l | 7.5 | 6.9 | 7.9 | 5.1 |
| M11 | 56 | 4, 3 | 8.6 | 7.4 | 8.1 | 5.1 |
| M12 | 54 | 5, 3 | 8.6 | 7.2 | 6.9 | 5.2 |
| M13 | 50 | 1, 3 | 8.8 | 7.1 | 8.4 | 5.1 |
| M14 | 41 | 1, 2 | 8.1 | 6.5 | 7.9 | 5.2 |
| M15 | 53 | 3, l | 8.9 | 7.3 | 9.1 | 5.0 |
| M16 | 57 | 5, 3 | 8.3 | 7.2 | 7.2 | 5.2 |
| M17 | 59 | 5, 3 | 8.3 | 7.1 | 8.1 | 5.0 |
| M18 | 52 | 3, l | 8.1 | 6.9 | 7.9 | 5.1 |
| F19 | 45 | 1, 2 | 7.9 | 6.5 | 7.2 | 5.1 |
| F20 | 56 | 5, 1 | 7.5 | 6.2 | 7.1 | 5.0 |
| F21 | 55 | 3, 1 | 7.9 | 5.6 | 6.8 | 5.1 |
| F22 | 57 | 4, 3 | 8.2 | 6.8 | 7.8 | 5.0 |
| F23 | 48 | 1, 2 | 8.5 | 7.2 | 8.2 | 5.2 |
| F24 | 51 | 3, l | 8.2 | 6.8 | 6.9 | 5.1 |
| F25 | 56 | 5, 3 | 8.5 | 6.9 | 8.1 | 5.2 |
| F26 | 55 | 5, 3 | 7.9 | 6.7 | 8.2 | 5.1 |
| F27 | 51 | 3, 1 | 7.8 | 6.2 | 7.6 | 5.1 |
| F28 | 52 | 3, 1 | 8.1 | 6.9 | 7.8 | 5.0 |
| F29 | 49 | 1, 2 | 7.3 | 5.9 | 7.1 | 5.0 |
| F30 | 51 | 1, 2 | 8.3 | 6.9 | 8.1 | 4.9 |

Code for drugs in Table 7: 1 = Metformin 500 mg/daily; 2 = Glipizide 10 mg/daily; 3 = Jardiance 10 mg/daily; 4 = Lantus Insulin Glargine 30 units/daily; 5 = Novolog Insulin Aspart 15 units twice a day. Fasting glucose is expressed in mmol/L; A1C is expressed in percent.

### Conclusions

The SOD gel tested here provides very strong antioxidants, diverse anti-inflammatory properties, enhances immunity and repair systems, has anti-infective capability, reduces oxidative stress caused by various ROS, and is able to improve cellular glucose uptake and slowly maintain blood glucose to acceptable level.

### Example 3: a Comparative Study of SOD Gel on Restoration of Skin in a Population Suffering from Acne Vulgaris

### Background and Objective

Acne vulgaris is a common chronic inflammatory skin disease of pilosebaceous units. It affects both male and females in different times of their lives, but more commonly in adolescents. It primarily affects the face, upper part of the chest, back, arm and thigh. This happens from a complex pathogenesis of overproduction of sebum by the sebaceous gland, clogging of hair follicles leading to formation of a plugged follicle, and accumulation of bacteria namely Propionibacterium. There then occurs an inflammatory reaction which leads to production of reactive oxygen species (ROS) by the damaged follicular walls. In addition, Propionibacterium bacteria produces some enzymes like lipases, protease, hyaluronidases which play an important role in the inflammatory process.

Oxygen, which is an important and vital component for life, can produce reactive species such as super-oxide anions and hydroxyl radicals. Super-oxide dismutase (SOD), catalase (CAT) and glucose 6 phosphate dehydrogenase are antioxidant enzymes that neutralize ROS. The SOD gel as described in Example 1 is a dietary supplement based on primary antioxidant superoxide dismutase, prebiotic fiber, diverse polyphenols from various fruit juices. It stimulates the immune system by activating T-cells, and acts as an antioxidant and anti-inflammatory product. It works on the cellular level to help to repair cell damage caused by free radicals. It also increases oxygen, reduces inflammation, and promotes skin well-being by promoting healthy digestion and gut flora.

The objective of this study is to evaluate the effect of SOD gel on a Bangladeshi population to restore skin well-being suffering from acne vulgaris.

### Study Design

A case and control study was conducted in different clinics in the Dhaka North City Corporation, Bangladesh. In total, 20 patients joined who were equally and randomly assigned in case and control groups i.e., 10 in each group.

Selection criteria were: male and female aged 20 years and above who were suffering from moderate to severe acne vulgaris with post inflammatory hyper-pigmentation, scars and uneven skin tone.

In the case group, study respondents used SOD gel along with oral doxycycline and tropical therapy, whereas the control group used only Oral Doxycycline and tropical therapy.

### Results

During the clinical assessment, it was found that the number and size of blemishes, level of inflammation and post inflammatory effect including hyperpigmentation and scars were reduced substantially and skin tone improved noticeably among the study respondents who used SOD gel along with oral doxycycline and tropical therapy compared to the control group.

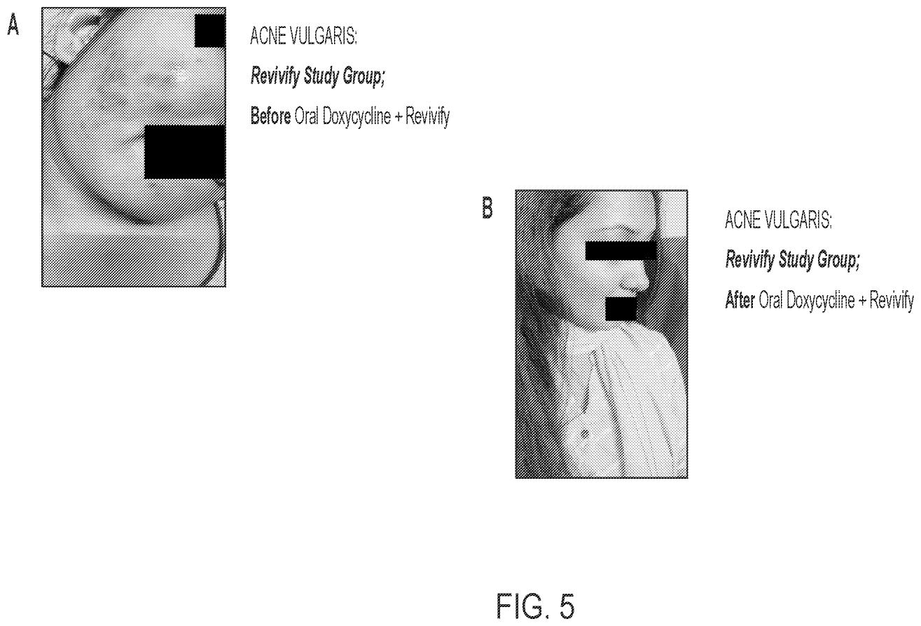

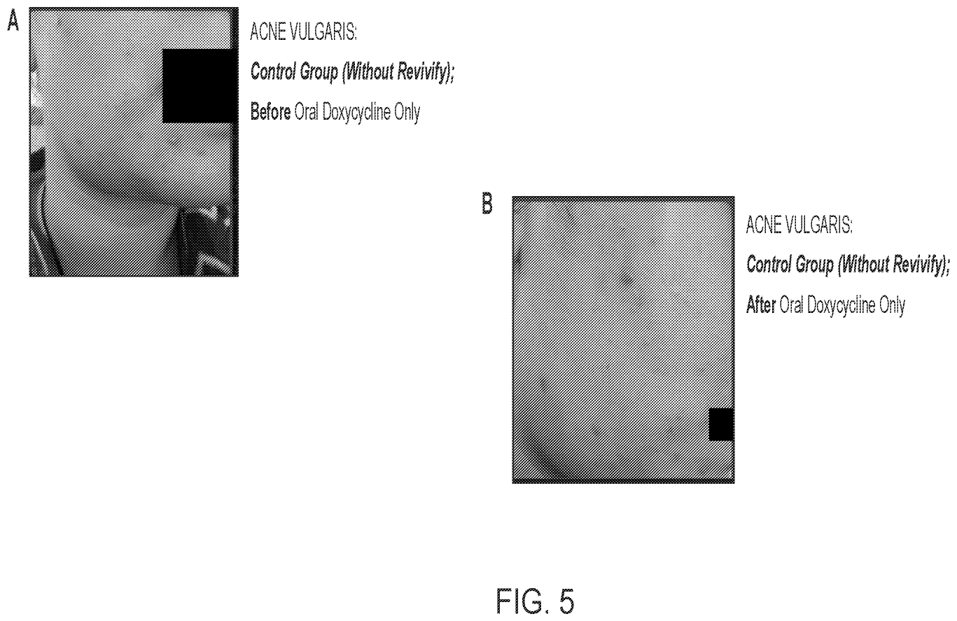

A shown in FIG. 5, a prior to treatment with SOD gel (REVIVIFY) and oral doxycycline, a subject had significant acne and pigmentation on the face (FIG. 5A). Following treatment, the acne and discoloration were significantly improved (FIG. 5B). A comparison from the control doxycycline only group is shown in FIG. 6. As can be seen, while there is some improvement with doxycycline treatment, significant acne and discoloration remain following treatment-compare FIG. 6A (before treatment) with FIG. 6B (after treatment).

### Conclusions

These findings suggest that SOD gel is useful to re-establish skin well-being among the Bangladeshi population. SOD gel may be a useful treatment option so that dermatologists can avoid over-use of antibiotics.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of maintaining tight junctions between cells in a tissue exposed to hypoxic conditions in a subject comprising administering to the subject a composition comprising:
a) from about 10 units to about 200 units superoxide dismutase; and
b) from about 500 mg to about 8000 mg soluble fiber.

2. The method of claim 1, wherein the tissue exposed to hypoxic conditions comprises the blood brain barrier.

3. The method of claim 1 or 2, wherein the tissue comprises human brain microvascular endothelial cells.

4. The method of any of claims 1 to 3, wherein the hypoxic conditions are caused by a stroke, heart attack or accident.

5. A method of preventing or reducing symptoms associated with a neurological disorder in a subject comprising administering to the subject a composition comprising:
a) from about 10 units to about 200 units superoxide dismutase; and
b) from about 500 mg to about 8000 mg soluble fiber.

6. The method of claim 6, wherein the neurological disorder is Parkinson Disease (PD), Multiple Sclerosis (MS), or Alzheimer's Disease (AD).

7. A method of reducing fasting glucose in a subject having diabetes comprising administering to the subject a composition comprising:
a) from about 10 units to about 200 units superoxide dismutase; and
b) from about 500 mg to about 8000 mg soluble fiber.

8. The method of claim 7, wherein the fasting glucose is reduced in the subject by at least about 30%.

9. A method of reducing hemoglobin A1C level in a subject having diabetes comprising administering to the subject a composition comprising:
a) from about 10 units to about 200 units superoxide dismutase; and
b) from about 500 mg to about 8000 mg soluble fiber.

10. The method of claim 9, wherein the hemoglobin A1C level is reduced by at least about 15%.

11. The method of any of claims 7 to 10, wherein the subject has type 2 diabetes.

12. A method of reducing acne symptoms in a subject comprising administering to the subject a composition comprising:
a) from about 10 units to about 200 units superoxide dismutase; and
b) from about 500 mg to about 8000 mg soluble fiber.

13. The method of claim 12, wherein the acne symptoms are comedos, pimples or skin discoloration.

14. The method of any one of claims 1 to 13, wherein the composition comprises from about 50 units to about 150 units superoxide dismutase.

15. The method of any one of claims 1 to 13, wherein the composition comprises from about 70 units to about 100 units superoxide dismutase.

16. The method of any one of claims 1 to 15, wherein the composition comprises from about 1000 mg to about 5000 mg soluble fiber.

17. The method of any one of claims 1 to 15, wherein the composition comprises from about 2000 mg to about 4000 mg soluble fiber.

18. The method of any one of claims 1 to 17, wherein the superoxide dismutase is extracted from melon, bovine liver, heterotrophic bacteria or marine phytoplankton.

19. The method of any one of claims 1 to 18, wherein the superoxide dismutase is a copper/zinc superoxide dismutase, an iron/manganese superoxide dismutase or a nickel superoxide dismutase.

20. The method of any one of claims 1 to 19, wherein the ratio of superoxide dismutase to soluble fiber in the composition is from about 1:100 to about 1:1000 by weight.

21. The method of any one of claims 1 to 19, wherein the ratio of superoxide dismutase to soluble fiber in the composition is from about 1:500 to about 1:700 by weight.

22. The method of any one of claims 1 to 21, wherein the soluble fiber is a water soluble polysaccharide.

23. The method of any one of claims 1 to 21, wherein the soluble fiber is selected from soluble corn fiber, inulin, dextrin, Guar gum, oligopolysaccharides, galactopolysaccharides fructo-oligosaccharides, lactulose, digestion-resistant starch, xylo-oligosaccharides and isomalto-oligossacharides.

24. The method of any one of claims 1 to 21, wherein the soluble fiber is soluble corn fiber.

25. The method of claim 24, wherein the soluble corn fiber is digestion-resistant maltodextrin.

26. The method of any one of claims 1 to 25, wherein the composition is in the form of a gel.

27. The method of any one of claims 1 to 25, wherein the composition is in the form of a liquid.

28. The method of any one of claims 1 to 25, wherein the composition is in the form of a powder.
